(19) United States
(12) Patent Application Publication
(10) Pub. No.: US 2007/0009980 A1
(43) Pub. Date: Jan. 11, 2007

(54) Continuous fluorogenic analyte assays with dendritic amplification of signal

(75) Inventor: Ronald J. Graham, San Ramon, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/142,617

(22) Filed: Jun. 1, 2005

Related U.S. Application Data
(60) Provisional application No. 60/576,320, filed on Jun. 1, 2004.

(51) Int. Cl. C12Q 1/37; C07D 487/22; C07D 417/14; C07D 405/14; C07D 403/14

(52) U.S. Cl. 435/23; 540/123; 548/181; 548/405; 548/464; 549/227; 977/754

(57) ABSTRACT

Substrate compounds comprising a trigger moiety, a reporter system comprising a plurality of fluorescent moieties, and a multivalent self-immolative dendrimer linker linking the trigger moiety to the reporter system.

### 1. CROSS-REFERENCES TO RELATED APPLICATIONS

This application claims benefit under 35 U.S.C. §119(e) to application Ser. No. 60/576,320, entitled “Continuous Fluorogenic Analyte Assays with Dendritic Amplification of Signal,” filed Jun. 1, 2004; the disclosure of which is incorporated herein by reference in its entirety.

### 2. FIELD

The present disclosure relates to fluorescent compositions and methods and kits for detecting and/or characterizing analytes, such as, for example, enzymes, and various uses thereof.

### 3. INTRODUCTION

A wide variety of assays are available for the detection and characterization of various analytes. For example, enzyme assays are important tools for studying and detecting enzymes for biological and industrial applications. In living organisms, enzymes perform a multitude of tasks, such as synthesis and replication of nucleic acids, modification, and degradation of polypeptides, synthesis of metabolites, and many other functions. Enzymes are also used in industry for many purposes, such as proteases used in laundry detergents, metabolic enzymes to make specialty chemicals such as amino acids and vitamins, and chirally specific enzymes to prepare enantiomerically pure drugs. In medical testing, enzymes are important indicators of the health or disease of human patients.

Although numerous approaches have been developed for assaying analytes, there is still a great need to find new assay designs that can be used to inexpensively and conveniently detect and characterize a wide variety of analytes. In the case of enzyme assays, the recent availability of a nearly complete sequence for the human genome has now made possible the identification of many enzyme candidates that will require years of research to uncover their various metabolic roles (see for example J. C. Venter et al., Science 291:1304-1351 (2001)). Such studies could be significantly facilitated by new assays that are suitable for high throughput screening. However, currently available assay protocols are inconvenient, expensive, or have other deficiencies.

### 4. SUMMARY

In one aspect, provided herein are substrate compounds useful for detecting and/or characterizing analytes, such as, for example, enzymes. The substrate compounds comprise a trigger moiety and a multivalent self-immolative dendrimer linker linking the trigger moiety and the reporter system. The fluorescence of the fluorescent moiety is quenched when in the substrate compound. The linker is capable of fragmenting to release at least one fluorescent moiety when the trigger moiety is acted upon by a triggering agent, thus leading to a detectable increase in a fluorescence signal. In some embodiments, triggering of the trigger moiety initiates an elimination reaction that results in the fragmentation of the linker to release the fluorescent moiety(ies), thereby increasing the fluorescent signal produced by the fluorescent moiety(ies).

In some embodiments, the reporter system comprises a plurality of fluorescent moieties. In some embodiments, the reporter system comprises a quenching moiety.

The trigger moiety can comprise any group that, when chemically altered (e.g., reduced or cleaved) by an analyte of interest, i.e. the “triggering agent”, results in fragmentation of the dendrimer linker. The chemical structure of the trigger moiety will depend, in part, upon the particular triggering agent. In some embodiments, the trigger moiety comprises a cleavage site for a cleaving enzyme. In these embodiments, the triggering agent can be any cleaving enzyme capable of cleaving the cleavage site under the condition of the assay. For example, the cleaving enzyme can be a lipase, an esterase, a phosphatase, a protease, a glycosidase, a carboxypeptidase or a catalytic antibody. In some embodiments, the triggering agent comprises a reducing agent (e.g., Zn and acetic acid), and the trigger moiety comprises a group capable of reduction (e.g., an aromatic nitro moiety or aromatic azide moiety). In some embodiments, the triggering agent comprises electromagnetic energy (such as light of a specific wavelength) and the trigger moiety comprises a photolabile group. In some embodiments, the triggering agent comprises an allyl deprotection agent and the trigger moiety comprises an allyl trigger group.

The fluorescent moiety in the reporter system can be any fluorescent entity that is operative in accordance with the various compositions and methods described herein. Non-limiting examples of fluorescent dyes that can comprise the fluorescent moiety include xanthene dyes such as fluorescein, sulfofluorescein and rhodamine dyes, cyanine dyes, bodipy dyes and squaraine dyes.

The reporter system has the property of being quenched when in the substrate compound. In some embodiments, the reporter system comprises a plurality of self-quenching fluorescent moieties. Triggering of the trigger moiety releases the fluorescent moieties from their close proximity, thereby unquenching their fluorescence. In some embodiments, the reporter system comprises at least one fluorescence-quencher moiety positioned within quenching proximity to at least one fluorescent moiety. Triggering of the trigger moiety by the triggering agent releases the quenching moiety from close proximity with the fluorescent moieties, thereby unquenching the fluorescence of the fluorescent moiety.

The mechanism by which the triggering leads to fragmentation is not critical. The fragmentation may involve a 1,4- 1,6- or a 1,8-elimination, for example.

The instant dendrimer compounds can be used in a variety of methods. In some embodiments, the dendrimer compounds are used to screen for, characterize and/or quantify, directly or indirectly, substrates, inhibitors, activators, or modulators of enzyme activity, as discussed further herein.

In other aspects, there are provided methods, e.g., for detecting an enzyme activity and for characterizing a potential enzyme modulator, using the disclosed compounds. In other aspects, the disclosure provides kits comprising the disclosed compounds.

These and other features of the various embodiments described herein will become more apparent from the detailed description.

### 5. BRIEF DESCRIPTION OF THE DRAWINGS

FIG. 1 provides a cartoon illustrating various self-immolative dendrimer substrate compounds.

FIG. 2 provides a cartoon illustrating a reaction of a self-immolative dendrimer substrate compound.

FIG. 3 depicts some embodiments of self-immolative dendrimer compounds.

FIG. 4 depicts an embodiment of a continuous fluorogenic enzyme assay utilizing a first generation self-immolative dendrimer substrate compound and showing the release of fluorescent moieties after enzyme mediated cleavage.

FIG. 5 depicts an embodiment of a continuous fluorogenic enzyme assay utilizing a second generation self-immolative dendrimer substrate compound.

FIG. 6 depicts the reduction of a nitroaromatic nitro trigger moiety in a self-immolative dendrimer compound.

FIG. 7A and FIG. 7B illustrate a scheme for the synthesis of an embodiment of self-immolative dendrimer substrate.

FIG. 8A and FIG. 8B illustrate reactions initiated by an enzyme or by a catalytic-antibody and that result in the generation of a π electron donor.

FIG. 9 depicts cleavage reactions of exemplary embodiments of a thermally labile trigger moiety and of a chemically labile trigger moiety.

### 6. DETAILED DESCRIPTION

It is to be understood that both the foregoing general description and the following detailed description are exemplary and explanatory only and are not restrictive of the various embodiments described herein. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

The present disclosure provides compositions, methods and kits that utilize certain fluorescently-labeled substrate compounds. The substrate compounds are capable of generating or providing a detectable fluorescent signal under specified conditions. The substrate compounds comprise a trigger moiety, a fluorescent reporter system, and a multivalent self-immolative dendrimer linker linking the trigger moiety to the reporter system. The fluorescent reporter system comprises at least one fluorescent moiety whose fluorescence increases when the substrate compound is reacted with a triggering agent of interest. Triggering of the trigger moiety leads to a cascade of self-elimination reactions, thereby releasing some or all of the fluorescent moieties, and results in an increase in fluorescence as described herein.

Advantageously, the substrate compounds described herein can be used in a continuous monitoring phase, in real time, to allow the user to rapidly determine whether a triggering agent activity is present in a sample, and optionally, the amount of triggering agent. In some embodiments, the trigger agent is an enzyme, and the substrate compounds can be used in a continuous monitoring phase, in real time, to allow the user to rapidly determine whether the enzyme activity is present in a sample, and optionally, the amount of specific activity of the enzyme.

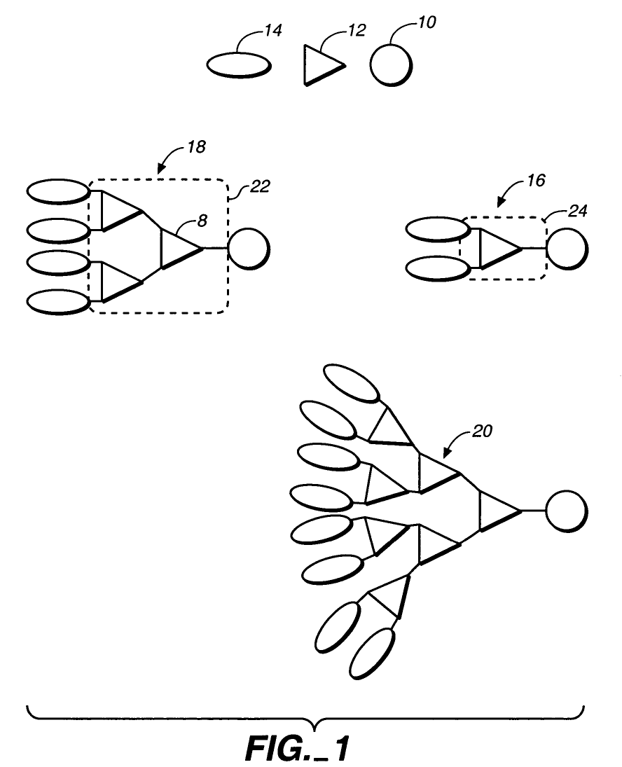

FIG. 1 graphically illustrates general features of some embodiments of substrate compounds disclosed herein, and shows a first generation (G1) self-immolative dendrimer compound (G1-SID) 16, a second generation (G2) self-immolative dendrimer compound (G2-SID) 18 and a third generation (G3) self-immolative dendrimer compound (G3-SID) 20. “Dendrimers” are well-defined, branched treelike molecules with multiple tail units (i.e., end groups). “Self-immolative dendrimers” are dendrimers that can release all of their tail units through a self-immolative chain fragmentation, which is initiated by a single triggering event (e.g., a cleavage event) at the dendrimer's core 8. The self-immolative compounds include a trigger moiety 10, one or more linker subunits 12 and tail units 14. In the compounds described herein, the tail units comprise the fluorescent reporter system, as described further herein below. A self-immolative dendrimer linker links the trigger moieties to the reporter system. Examples of such a linker are schematically shown within the dashed lines at 22 and at 24.

The self-immolative dendrimer linker of the present disclosure can be selected so as to link the trigger moiety to more than two tail units, in the case of a G1-SID, or to more than two linker subunits in the case of a Gn-SID, thus rendering the number of ramifications (i.e., branching multiplicity) of the dendrimer of the present disclosure being, in some embodiments, between 2 and 5 or between 2 and 3.

As is described herein, the self-immolative chemical linker of the present disclosure is selected such that it undergoes a sequence of self-immolative reactions upon cleavage of the trigger moiety. Self-immolative reactions typically involve electronic cascade self-elimination and therefore self-immolative systems typically include electronic cascade units which self-eliminate through, for example, linear or cyclic 1,4-elimination, 1,6-elimination, etc. (see, e.g., WO 02/083180). The dendrimer substrate compounds of the present disclosure can be designed such that the self-immolative chemical linker undergoes electronic cascade self-elimination to thereby release two or more end groups. Such chemical linkers can be based on a multifunctional aromatic unit which can be linked to both the trigger moiety and to two or more tail units, or other chemical linkers and can further be subjected to electronic cascade self-elimination.

The dendrimer substrate compound can comprise various numbers of tail units. In FIG. 1, the number of tail units 14 in compounds 16, 18 and 20 is 2, 4 and 8, respectively. In general, the number of tail units, n, is equal to N_{b}^{G}, where N_{b} is the branching multiplicity, and G is the generation. In the embodiments illustrated in FIG. 1, N_{b} is 2. In some embodiments, Nb is a value in the range of 2-5. The number of tail units, n, will depend upon the structure of the linker subunit (embodiments of which are described herein below) and the selected value of G, and may be any value as long as the dendrimer compound is operative in the accordance with the various methods as described herein. In some embodiments, G is in the range of 2-6, 1-10, 1-20, 1-50 or 1-100.

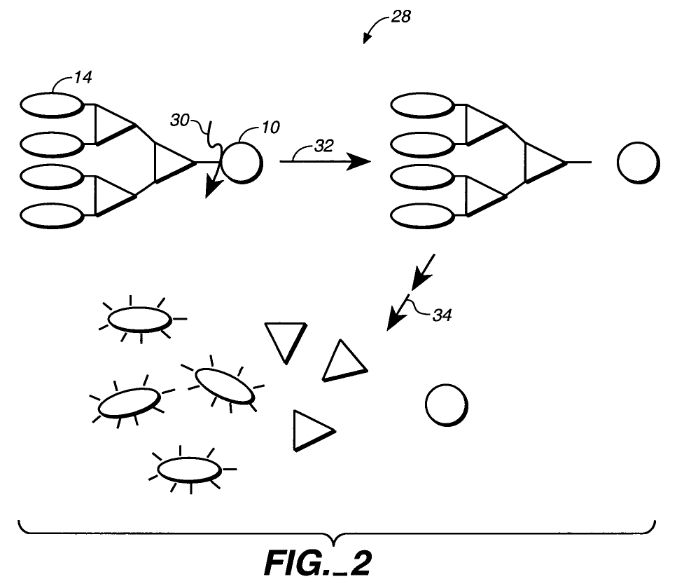

In general, in the self-immolative dendrimer compounds as described herein, a triggering agent causes a molecular change in a trigger moiety. In some embodiments, the molecular change results in release of the trigger moiety. FIG. 2 graphically illustrates a self-immolative chain fragmentation process 28 in which a molecular change in the trigger moiety 10 results in cleavage 30 and allows release 32 of the trigger moiety. The dendrimer collapses into separate monomeric building blocks after a single triggering step that triggers a cascade of self-elimination reactions 34, thereby releasing the tail units 14 from the periphery of the substrate.

In the substrate compounds described herein, the tail units 14 comprise a fluorescent reporter system. In some embodiments, all of the tail units in the reporter system are identical, and each comprises a fluorescent moiety. In some embodiments, the reporter system comprises tail units that are not all identical. In some embodiments, the tail units comprise quencher moieties and fluorescent moieties, as described below. The fluorescence of the fluorescent moieties in the reporter system of the dendrimer substrate compound is at least partially quenched. In the embodiment shown in FIG. 2, the tail units are all identical and are at least partially quenched when incorporated into the substrate compound.

The quenching may be accomplished by a variety of different mechanisms. In some embodiments, the reporter system comprises fluorescent moieties that are capable of “self-quenching” when in sufficiently close proximity to another fluorescent moiety of the same type. Triggering of the trigger moiety releases the fluorescent moieties from close proximity by fragmentation of the substrate compound, thereby unquenching the fluorescence of the fluorescence moiety.

“Quench” has its standard meaning and is intended to refer to a measurable reduction in the fluorescence intensity of a fluorescent group or moiety as measured at a specified wavelength, regardless of the mechanism by which the reduction is achieved. As specific examples, the quenching may be due to molecular collision, energy transfer such as FRET, a change in the fluorescence spectrum (color) of the fluorescent group or moiety or any other mechanism (or combination of mechanisms). The quenching may be due to, for example, photoinduced electron transfer (PET). The amount of the reduction is not critical and may vary over a broad range. The only requirement is that an increase in fluorescence be measurable by the detection system being used. In some embodiments, the fluorescence of the fluorescent moiety is fully, or at least partially, quenched when the fluorescent moiety is incorporated into the reporter system of the substrate compound. In some embodiments of the substrate compounds, the fluorescence signal of the fluorescent moieties in a fluorescent reporter system is quenched by at least 10%, 20%, 30%, 40%, 50%, 60%, 70%, 80% or 90%. In some embodiments, the fluorescence signal of the fluorescent moieties in a reporter system is quenched by 100%. In some embodiments, the substrate is designed, as described herein, such that fluorescence of the resultant products of the reaction is at least 2 times, at least 3 times, at least 4 times, at least 5 times (or more) the fluorescence of the substrate, on a mole:mole basis.

The fluorescent moiety of the reporter system of the self-immolative dendrimer substrate may be any fluorescent entity that is operative in accordance with the compositions and methods described herein. Typically, the fluorescent moiety comprises a fluorescent dye that in turn comprises a resonance-delocalized system or aromatic ring system that absorbs light at a first wavelength and emits fluorescent light at a second wavelength in response to the absorption event. The fluorescent moiety may be any entity that provides a fluorescent signal that can be used to follow triggering of a self-immolative dendrimer compound as described herein. The fluorescent moiety may be any fluorescent dye capable of producing a detectable fluorescence signal in the assay medium.

In some embodiments, the reporter system comprises at least one fluorescent moiety. In some embodiments, the reporter system comprises two or more fluorescent moieties. Non-limiting examples of suitable fluorescent dyes that can comprise the fluorescent moiety(ies) include xanthene dyes such as fluorescein, sulfofluorescein and rhodamine dyes, cyanine dyes, bodipy dyes and squaraine dyes. Fluorescent moieties comprising other fluorescent dyes may also be used. In some embodiments, a fluorescent moiety comprises a fluorescein dye.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

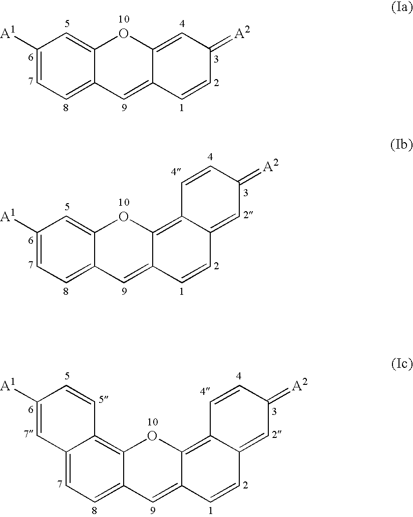

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In one embodiment, typical substituents comprise, but are not limited to, —H, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each H is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

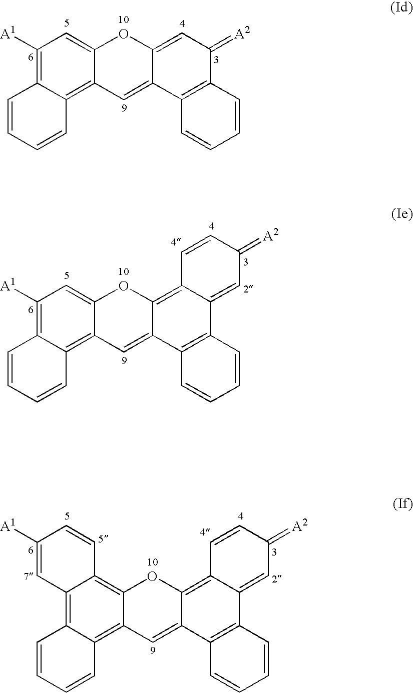

The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A^{1 l is NH}₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens are illustrated below:

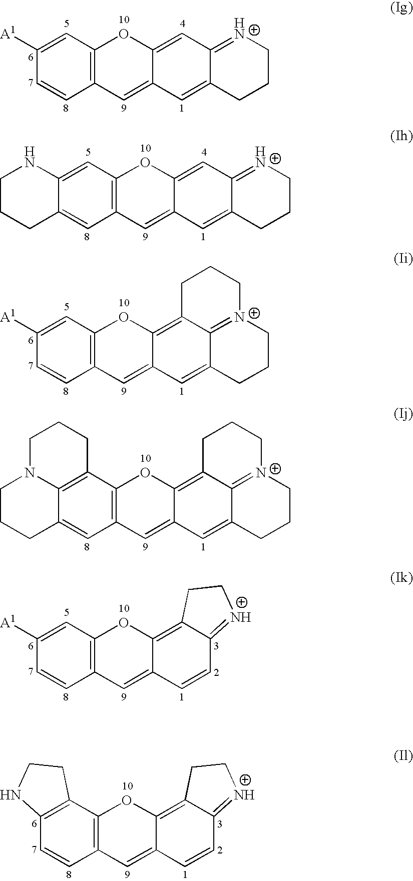

The parent ring may also comprise a substituent at the C9 position. In some embodiments, the C9 substituent is selected from acetylene, lower (e.g., from 1 to 6 carbon atoms) alkanyl, lower alkenyl, cyano, (C₂-C₁₀) aryl, phenyl, (C₂-C₁₀) heteroaryl, (C₂-C₁₀) electron-rich heteroaryl and substituted forms of any of the preceding groups. In embodiments in which the parent ring comprises benzo or aryleno bridges fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures:

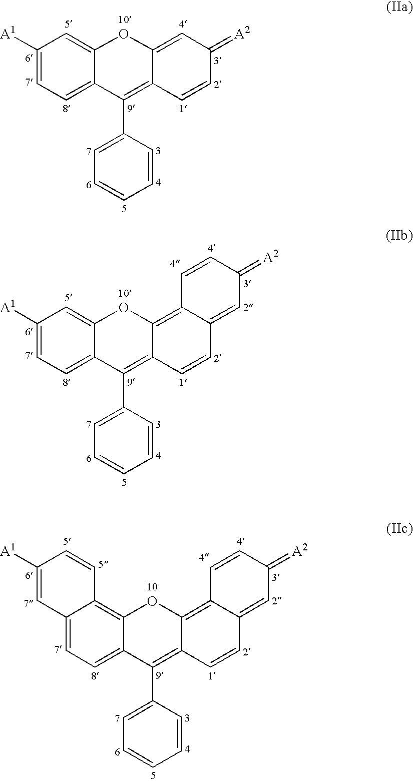

The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FüR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In one embodiment, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In one embodiment, the fluorescein moiety comprises a 4,7-dichloro-orthocarboxyfluorescein dye.

In some embodiments, the fluorescent moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent reporter system can comprise dyes that operate cooperatively with one another such as, for example by FRET or another mechanism, to provide large Stoke's shifts. Such a reporter system can comprise a fluorescence donor moiety and a fluorescence acceptor moiety, and may comprise additional moieties that act as both fluorescence acceptors and donors. The fluorescence donor and acceptor moieties can comprise any of the previously described dyes, for example, provided that dyes are selected that can act cooperatively with one another. In a specific embodiment, the fluorescent reporter system comprises a fluorescence donor moiety which comprises a fluorescein dye and a fluorescence acceptor moiety which comprises a fluorescein or rhodamine dye. Non-limiting examples of suitable dye pairs are described in U.S. Pat. Nos. 6,399,392, 6,232,075, 5,863,727, and 5,800,996. In some embodiments, the fluorescent reporter system does not include pyrene.

In some embodiments, fluorescence quenching in the instant substrate compounds can be achieved by use of a reporter system that comprises at least one quenching moiety. The quenching moiety can be any moiety capable of quenching the fluorescence of a fluorescent moiety when it is in suitable proximity thereto, such as for example, by orbital overlap (formation of a ground state dark complex), collisional quenching, FRET (e.g., in the range of about 10-100 angstrom), photoinduced electron transfer (PET), or another mechanism or combination of mechanisms. The quenching moiety can itself be fluorescent, or it can be non-fluorescent. In some embodiments, the quenching moiety comprises a fluorescent dye that has an absorbance spectrum that sufficiently overlaps the emission spectrum of the fluorescent moiety of the substrate compound such that it quenches the fluorescence of the fluorescent moiety when in close proximity thereto. In such embodiments, selecting a quenching moiety that fluoresces at a wavelength resolvable from that of the fluorescent moiety can provide an internal signal standard to which the fluorescence signal can be referenced. In some embodiments, a reporter system includes one or more quenching moieties, and triggering of the trigger moiety releases the quenching and fluorescent moieties from close proximity, thereby unquenching the fluorescence of the fluorescence moiety.

The quenching moiety, when present, can include any moiety capable of quenching the fluorescence of the fluorescent moiety of the enzyme substrate used in the assay (or one or more of the substrates if a plurality of substrates are used). Compounds capable of quenching the fluorescence of the various different types of fluorescent dyes discussed above, such as xanthene, fluorescein, rhodamine, cyanine, phthalocyanine and squaraine dyes, are well-known. Such quenching compounds can be non-fluorescent (also referred to as “dark quenchers” or “black hole quenchers”) or, alternatively, they may themselves be fluorescent. Examples of suitable non-fluorescent dark quenchers that can comprise the quenching moiety include, but are not limited to, Dabcyl, Dabsyl, the various non-fluorescent quenchers described in U.S. Pat. No. 6,080,868 (Lee et al.) and the various non-fluorescent quenchers described in WO 03/019145 (Ewing et al.). Examples of suitable fluorescent quenchers include, but are not limited to, the various fluorescent dyes described above.

The ability of a quenching moiety to quench the fluorescence of a particular fluorescent moiety may depend upon a variety of different factors, such as the mechanisms of action by which the quenching occurs. The mechanism of the quenching is not critical to success, and may occur, for example, by collision, by FRET, by another mechanisms or combination of mechanisms. The selection of a quencher for a particular application can be readily determined empirically. As a specific example, the dark quencher Dabcyl and the fluorescent quencher TAMRA have been shown to effectively quench the fluorescence of a variety of different fluorophores. In a specific embodiment, a quencher can be selected based upon its spectral overlap properties spectral overlap with the fluorescent moiety. For example, a quencher can be selected that has an absorbance spectrum that sufficiently overlaps the emission spectrum of the fluorescent moiety such that the quencher quenches the fluorescence of the fluorescent moiety are in close proximity to one another, such as when the quencher molecule and substrate including the quencher moiety are integrated into the substrate.

Regardless of the mechanism by which the quenching effect is achieved, fragmentation of the substrate compound leads to unquenching of the fluorescence signal, thereby producing a detectable increase in fluorescence. The mechanism by which the fragmentation occurs is not critical, and can be selected by the user, depending in part, on the particular application. For example, fragmentation may involve a 1,6-, 1,4-, or 1,8-elimination reaction, leading to the release of the fluorescent moiety (and quenching moiety, if present) from the reporter system as a result of fragmentation of the self-immolative dendrimer linker.

A fluorescent dye (or quencher) can be synthesized (or purchased) which includes a reactive group for coupling of the dye to a dendrimer linker as described herein. Such a dye (or quencher) can be can be reacted with a complementary functionality in an intermediate during the synthesis of a self-immolative dendrimer substrate compound.

The point of attachment of a reactive group to the fluorescent dye (or quencher) can be any position that does not interfere with the ability of the substrate compound that is formed to function as a substrate as described herein. In some embodiments, for xanthene dyes, a reactive group can be attached to C9 phenyl (if present), a rhodamine exocyclic ring nitrogen, or via a 4-aminomethyl group.

Examples of complementary functional groups include pairs such as a nucleophilic functional group and an electrophilic function group. Examples of nucleophilic functional groups include alcohols, alkoxides, amines, hydroxylamines, and thiols. Examples of electrophilic functional groups include succinimidyl ester, isothiocyanate, sulfonyl chloride, sulfonate ester, silyl halide, 2,6-dichlorotriazynyl, pentafluorophenyl ester, phosphoramidite, maleimide, iodoacetamide, haloacetyl, epoxide, alkylhalide, allyl halide, aldehyde, ketone, acylazide, anhydride, and activated esters such as NHS ester. Additional examples of nucleophilic and electrophilic functional groups are described in U.S. Pat. Nos. 6,248,884, 5,231,191 and 5,227,487.

In the instant substrate compounds, the fluorescent moiety(ies) and/or quenching moiety(ies) of the reporter system and the self-immolative dendrimer linker can be attached in any way that permits them to perform their respective functions, and can be attached via an optional linkage. The chemical composition of a linkage between the fluorescent moiety (or quenching moiety) and the self-immolative dendrimer linker of the substrate compound is not critical for success. Choosing a linkage having properties suitable for a particular application is within the capabilities of those having skill in the art. The linkage can comprise any combination of atoms that will function to connect the self-immolative dendrimer linker with the fluorescent moiety (or quenching moiety) but will not interfere with an enzyme assay as described herein.

The length and chemical composition of the linkage can be selectively varied. In some embodiments, the linkage can be selected to have specified properties. For example, the linkage can be hydrophobic in character, hydrophilic in character, long or short, rigid, semirigid or flexible, depending upon the particular application. The linkage can be optionally substituted with one or more substituents or one or more groups for the attachment of additional substituents, which may be the same or different, thereby providing a “polyvalent” capable of conjugating additional molecules or substances to the molecule. In certain embodiments, however, the linkage does not comprise such additional substituents.

A wide variety of linkages comprised of stable bonds that are suitable for use in the substrates described herein are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, the linkage can include single, double, triple or aromatic carbon-carbon bonds, nitrogen-nitrogen bonds, carbon-nitrogen bonds, carbon-oxygen bonds, carbon-sulfur bonds and combinations of such bonds, and may therefore include functionalities such as carbonyls, ethers, thioethers, carboxamides, sulfonamides, ureas, urethanes, hydrazines, etc. In some embodiments, the linkage comprises from 1-20 non-hydrogen atoms selected from the group consisting of C, N, O, and S and is composed of any combination of ether, thioether, amine, ester, carboxamide, sulfonamides, hydrazide, aromatic and heteroaromatic groups.

Choosing a linkage having properties suitable for a particular application is within the capabilities of those having skill in the art. For example, where a rigid linkage is desired, it may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible linkage is desired, it may comprise a flexible polypeptide such as polyglycine or a flexible saturated alkanyldiyl or heteroalkanyldiyl. Hydrophilic linkages may comprise, for example, polyalcohols or polyethers such as polyalkyleneglycols, or other spacers. Hydrophobic linkages may comprise, for example, alkyldiyls or aryldiyls.

In some embodiments, the linkages are formed from pairs of complementary reactive groups capable of forming covalent bonds with one another. “Complementary” nucleophilic and electrophilic groups (or precursors thereof that can be suitably activated) useful for effecting bonds stable to biological and other assay conditions are well known. Examples of suitable complementary nucleophilic and electrophilic groups, as well as the resultant linkages formed therefrom, are provided in Table 1.

TABLE 1

| Electrophilic Group | Nucleophilic Group | Resultant Covalent Linkage |
|---|---|---|
| activated esters* | Amines/anilines | carboxamides |
| Acyl azides** | Amines/anilines | carboxamides |
| Acyl halides | Amines/anilines | carboxamides |
| Acyl halides | alcohols/phenols | esters |
| Acyl nitriles | alcohols/phenols | esters |
| Acyl nitriles | Amines/anilines | carboxamides |
| aldehydes | Amines/anilines | imines |
| aldehydes or ketones | Hydrazines | hydrazones |
| aldehydes or ketones | Hydroxylamines | oximes |
| alkyl halides | Amines/anilines | alkyl amines |
| alkyl halides | carboxylic acids | esters |
| alkyl halides | Thiols | thioethers |
| alkyl halides | alcohols/phenols | ethers |
| alkyl sulfonates | Thiols | thioethers |
| alkyl sulfonates | carboxylic acids | esters |
| alkyl sulfonates | alcohols/phenols | esters |
| anhydrides | alcohols/phenols | esters |
| anhydrides | Amines/anilines | caroboxamides |
| Aryl halides | Thiols | thiophenols |
| Aryl halides | Amines | aryl amines |
| aziridines | Thiols | thioethers |
| boronates | Glycols | boronate esters |
| carboxylic acids | Amines/anilines | carboxamides |
| carboxylic acids | Alcohols | esters |
| carboxylic acids | Hydrazines | hydrazides |
| carbodiimides | carboxylic acids | N-acylureas or anhydrides |
| diazoalkanes | carboxylic acids | esters |
| epoxides | Thiols | thioethers |
| haloacetamides | Thiols | thioethers |
| halotriazines | Amines/anilines | aminotriazines |
| halotriazines | alcohols/phenols | triazinyl ethers |
| imido esters | Amines/anilines | amidines |
| isocyanates | Amines/anilines | ureas |
| isocyanates | alcohols/phenols | urethanes |
| isothiocyanates | Amines/anilines | thioureas |
| maleimides | Thiols | thioethers |
| phosphoramidites | Alcohols | phosphate esters |
| Silyl halides | Alcohols | silyl ethers |
| sulfonate esters | Amines/anilines | alkyl amines |
| sulfonate esters | Thiols | thioethers |
| sulfonate esters | carboxylic acids | esters |
| sulfonate esters | Alcohols | esters |
| sulfonyl halides | Amines/anilines | sulfonamides |
| sulfonyl halides | phenols/alcohols | sulfonate esters |

*Activated esters, as understood in the art, generally have the formula —C(O)Z, where Z is, a good leaving group (e.g., oxysuccinimidyl, oxysulfosuccinimidyl, 1-oxybenzotriazolyl, etc.).
**Acyl azides can rearrange to isocyanates.

The substrate compounds described herein can be readily formed by synthetic methods known in the art. Fluorescent dyes that can be used to prepare the substrate compound can be prepared synthetically using conventional methods or purchased commercially (e.g. Sigma-Aldrich and/or Molecular Probes). Non-limiting examples of suitably reactive fluorescent dyes that are commercially available from Molecular Probes (Eugene, Oreg.) are provided in Table 2, below:

TABLE 2

| Catalog | |
|---|---|
| Number | Product Name |
| A1351 | 4′-(aminomethyl)fluorescein, hydrochloride |
| A1353 | 5-(aminomethyl)fluorescein, hydrochloride |
| C-20050 | 5-carboxyfluorescein-bis-(5-carboxymethoxy-2-nitrobenzyl) |
| | ether, -alanine-carboxamide, succinimidyl ester (CMNB-caged |
| | carboxyfluorescein, SE) |
| C-2210 | 5-carboxyfluorescein, succinimidyl ester (5-FAM, SE) |
| C-1311 | 5-(and-6)-carboxyfluorescein, succinimidyl |
| | ester (5(6)-FAM, SE) |
| D-16 | 5-(4,6-dichlorotriazinyl) aminofluorescein (5-DTAF) |
| F-6106 | 6-(fluorescein-5-carboxamido)hexanoic acid, succinimidyl |
| | ester (5-SFX) |
| F-2182 | 6-(fluorescein-5-(and-6)-carboxamido) hexanoic acid, |
| | succinimidyl ester (5(6)-SFX) |
| F-6129 | 6-(fluorescein-5-(and-6)-carboxamido) hexanoic acid, |
| | succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

An example of a synthetic scheme for a substrate comprising an enzyme cleavable trigger moiety is provided in Example 1.

As indicated above, the instant substrate compounds comprise a trigger moiety. Without intending to be bound by any theory of operation, it is believed that the trigger moiety can comprise any moiety that when acted on by a molecule (or reaction condition) of interest, i.e., the “triggering agent”, is capable of generating an intermediate that spontaneously rearranges resulting in fragmentation of the substrate compound.

The chemical structure of the trigger moiety will depend, in part, upon the particular triggering agent to be detected. In some embodiments, the triggering agent is an enzyme and the trigger moiety comprises a cleavage site for a cleaving enzyme. In these embodiments, the triggering agent can be any cleaving enzyme capable of cleaving the cleavage site under the conditions of the assay. The chemical composition of the trigger moiety will depend upon, among other factors, the requirements of the cleaving enzyme. The analysis of a particular enzyme activity requires the use of a substrate compound that will be recognized and altered by one or more enzymes that exhibit that activity. Thus, the trigger moiety can comprise a leaving group selected for recognition and removal (e.g., via hydrolytic cleavage) by the enzyme to be analyzed. For example, if the cleaving enzyme is a protease, the trigger moiety can comprise a peptide (or analog thereof) recognized and cleaved by the particular protease. If the cleaving enzyme is a nuclease, the trigger moiety can comprise an oligonucleotide (or analog thereof) recognized and cleaved by a particular nuclease. If the cleaving enzyme is glycosidase, the trigger moiety can comprise a carbohydrate recognized and cleaved by a particular glycosidase. In some embodiments, the leaving group can be selected from amino acids, peptides, saccharides, sulfates, phosphates, esters, phosphate esters, nucleotides, polynucleotides, nucleic acids, pyrimidines, purines, nucleosides, and peptides. Other leaving groups suitable for an enzyme to be assayed can be determined empirically or obtained from the literature.

Sequences and structures recognized and cleaved by the various different types of cleaving enzymes are well known. Any of these sequences and structures can comprise the trigger moiety. Although the cleavage can be sequence specific, in some embodiments it can be non-specific. For example, the cleavage can be achieved through the use of a non-sequence specific nuclease, such as, for example, an RNase.

In some embodiments, the trigger moiety comprises a cleavage site comprising a phosphate moiety that is capable of being hydrolyzed by a phosphatase. The trigger moiety may also comprise additional residues that facilitate specificity, affinity and/or rate of hydrolysis of the particular phosphatase. The trigger moiety may be designed to be recognized by a particular phosphatase or group of phosphatases. For example, a cleavage site comprising a phosphate group capable of being cleaved by a phosphatase could be used as trigger moiety and the corresponding phosphatase used as the specified trigger agents (see, e.g., Zhu, et al., BIOORG. MED. CHEM. LETT. 10:1121-1124 (2000), and Ueda, et al., BIOORG. MED. CHEM. LETT. 8:1761-1766 (1993)).

In some embodiments, the trigger moiety comprises a cleavage site comprising one or more carbohydrates that are capable of being hydrolyzed by a glycosidase, such as β-galactosidase or β-glucoronidase. The trigger moiety may also comprise additional residues that facilitate specificity, affinity and/or rate of hydrolysis of the particular glycosidase. The trigger moiety may be designed to be recognized and hydrolyzed by a particular glycosidase or group of glycosidases. Structures recognized and cleaved by glycosidases are also well known (see, e.g., Florent, et al., J. MED. CHEM. 41:3572-3581 (1998), Ghosh, et al., TETRAHEDRON LETTERS 41:4871-4874 (2000), Michel, et al., ATTA-UR-RAHMAN (ED) 21:157-180 (2000), and Leu, et al., J. MED. CHEM. 42:3623-3628 (1999)).

In some embodiments, the trigger moiety comprises a cleavage site comprising uncharged esters of glycerol and fatty acids that are capable of being hydrolyzed by a lipase, such as triacylglycerol lipase. The trigger moiety may also comprise additional residues that facilitate specificity, affinity and/or rate of hydrolysis of the particular lipase. The trigger moiety may be designed to be recognized and hydrolyzed by a particular lipase or group of lipases. Structures recognized and cleaved by lipases and esterases are also well known (see, e.g., Ohwada, et al., BIOORG. MED. CHEM. LETT. 12:2775-2780 (2002), Sauerbrei, et al., ANGEW. CHEM. INT. ED. 37:1143-1146 (1998), Greenwald, et al., J. MED. CHEM. LETT. 43:475-487 (2000), Dillon, et al., BIOORG. MED. CHEM. LETT. 14:1653-1656 (1996), and Greenwald, et al., J. MED. CHEM. 47:726-734 (2004)). Specific examples of substrate compounds comprising trigger moieties cleavable by lipases and esterases are described in more detail below.

In some embodiments, the trigger moiety comprises a cleavage site comprising an ester moiety that is capable of being hydrolyzed by an esterase. The trigger moiety may also comprise additional residues that facilitate specificity, affinity and/or rate of hydrolysis of the particular esterase. The trigger moiety may be designed to be recognized and hydrolyzed by a particular esterase or group of esterases.

In some embodiments, the trigger moiety comprises a cleavage site comprising a peptide bond, a peptide analog, or a peptide sequence that is capable of being hydrolyzed by a protease, such as carboxypeptidase A, carboxypeptidase G2, protease plasmin, trypsin, proteases such as serine, cysteine, aspartyl and metalloproteases. For example, in some embodiments the trigger moiety comprises a peptide sequence or peptide analog that is recognized and cleaved by a protease. In other embodiments, the trigger moiety comprises cleavage site comprising an amidic, urethanic, or ureidic bond linking the multivalent linker to an amino acid. The trigger moiety may also comprise additional residues that facilitate specificity, affinity and/or rate of hydrolysis of the particular protease. The trigger moiety may be designed to be recognized and hydrolyzed by a particular protease or group of proteases. Structures recognized and cleaved by proteases/proteolytic enzymes are also well known (see, e.g., Niculescu-Duvaz, et al., J. MED. CHEM. 41:5297-5309 (1998), Niculescu-Duvaz, et al., J. MED. CHEM. 42:2485-2489 (1999), Greenwald, et al., J. MED. CHEM. 42:3657-3667 (1999), de Groot, et al., BIOORG. MED. CHEM. LETT. 12:2371-2376 (2002), Dubowchik, et al., BIOCONJUGATE CHEM. 13:855-869 (2002), and de Groot, et al., J. ORG. CHEM. 66:8815-8830 (2001)). Specific examples of substrate compounds comprise trigger moieties cleavable by protease plasmin, trypsin, and carboxypeptidase G2.

In some embodiments, the trigger moiety comprises a cleavage site comprising a transition state analogue to which a catalytic antibody has been raised. For example, N-methylcarbamate can be attached to a carrier protein and used as a transition state analogue to which catalytic antibodies can be raised. Hydrolysis of N-methylcarbamate by the catalytic antibody results in fragmentation of the self-immolative dendrimer linker and release of the fluorescent moiety(ies) from the reporter system. The trigger moiety may also comprise additional residues that facilitate specificity, affinity and/or rate of hydrolysis of the particular catalytic antibody.

In the exemplary substrate compounds 16, 18 or 20, trigger moiety 10 can comprise a cleavage site for a cleaving enzyme, and an electron donor group, such as —O—, —NH—, or —S— attached to, for example, the carbon atom at position C1 of a benzyl backbone. The trigger moiety may include a spacer (i.e., additional linkage) that facilitates the attachment of the cleavage site to the substrate compound. In embodiments comprising a spacer, the spacer is capable of undergoing spontaneous rearrangement such that fragmentation of the self-immolative dendrimer linker results. Examples of cleavage sites and cleaving enzymes, as well as spacers, are provided in Table 3.

TABLE 3

| | Cleavage Site with optional | |
|---|---|---|
| Cleavage Site | spacer | Cleaving Enzyme |
| 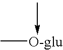 | 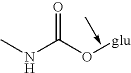 | β-glucuronidase |
| 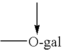 | 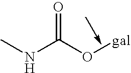 | β-galactosidase |
| 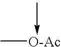 | | lipase/esterase |
| 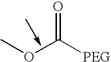 | 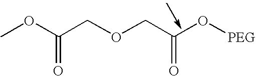 | lipase/esterase |
| 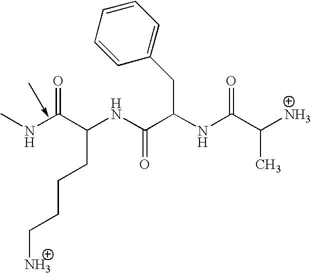 | | protease plasmin |
| 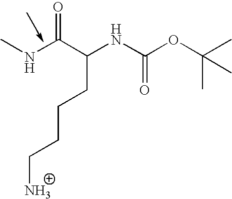 | | trypsin |
| 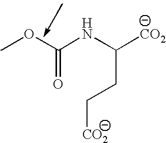 | | carboxypeptidase G2 |
| 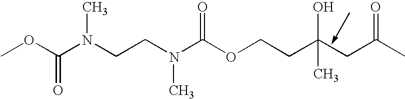 | | catalytic antibody |
| 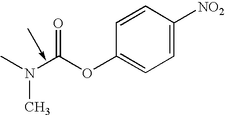 | | catalytic antibody |

(Glu and gal represent the carbohydrates glucoronide and galactose, respectively. The cleavage site is indicated by an arrow.)

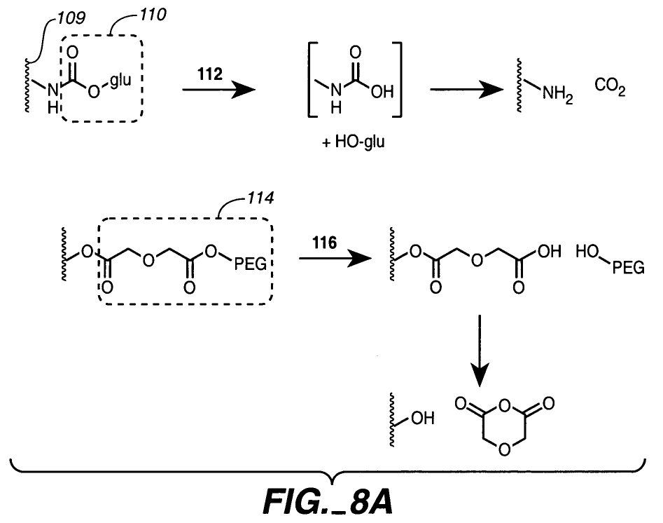

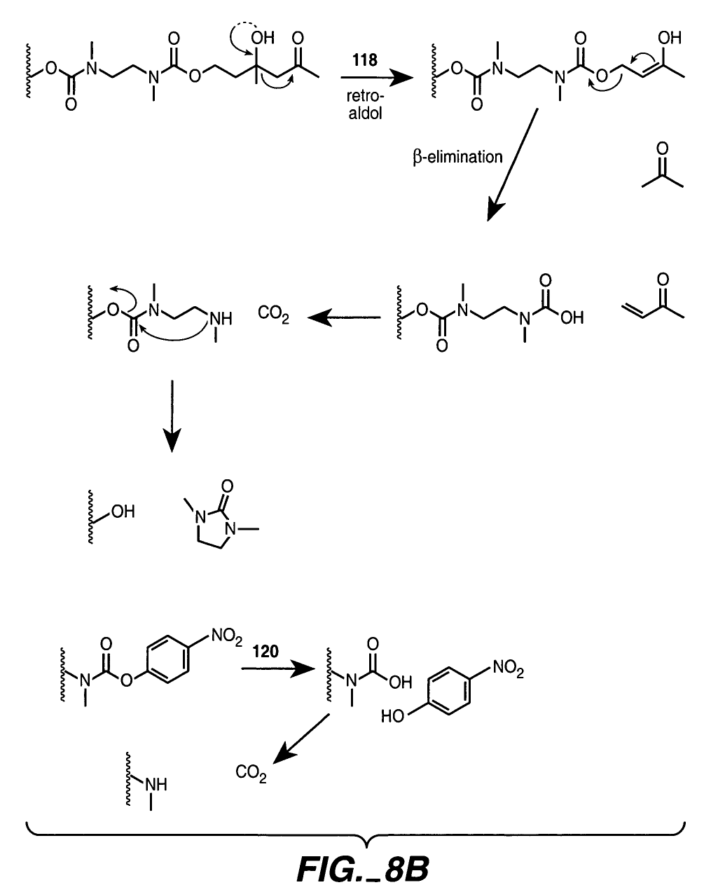

As will be appreciated by a person of skill in the art, the illustrated cleavage sites, cleavage sites with optional spacers, and cleaving enzymes are merely exemplary trigger moieties and triggering agents. Any trigger moiety comprising a cleavage site suitable for cleavage by a cleavage enzyme and that can be appropriately cleaved to unmask a π electron donor, including but not limited to, —O— or —NH—, could be used. FIG. 8A and FIG. 8B illustrate reactions initiated by β-glucuronidase 112, by an esterase 116, by a catalytic antibody 118, and by another catalytic antibody 120. Examples of trigger moieties are shown at 110 and at 114. In the figures, the remainder of a self-immolative dendrimer compound is represented by a wavy line such as shown at 109.

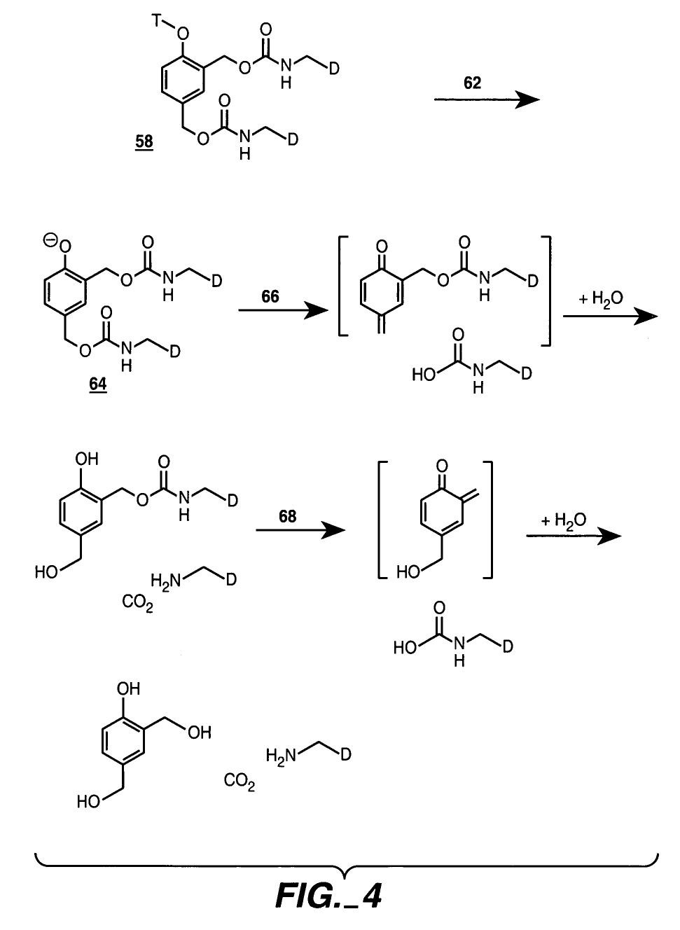

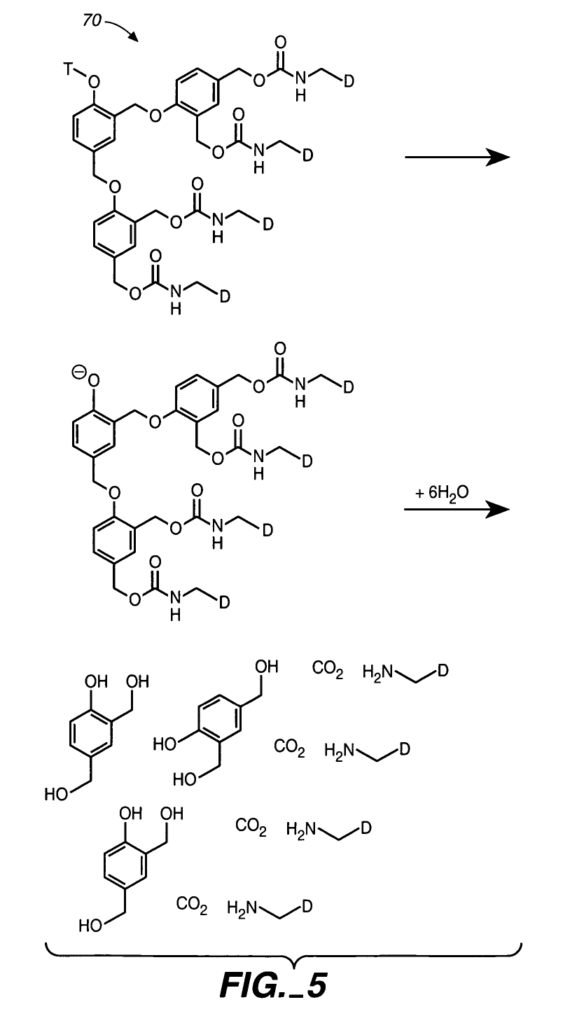

FIG. 4 shows an embodiment of a first generation dendrimer substrate compound 58 in which “T” is a leaving group recognized by an enzyme. Cleavage of “T” by an enzyme yields phenoxide 64. An initial 1,4-elimination reaction 66 is followed by a second 1,4-elimination reaction 68, and results in the release of fluorescent moieties, D. FIG. 5 illustrates a similar reaction starting from second generation dendrimer substrate compound 70.

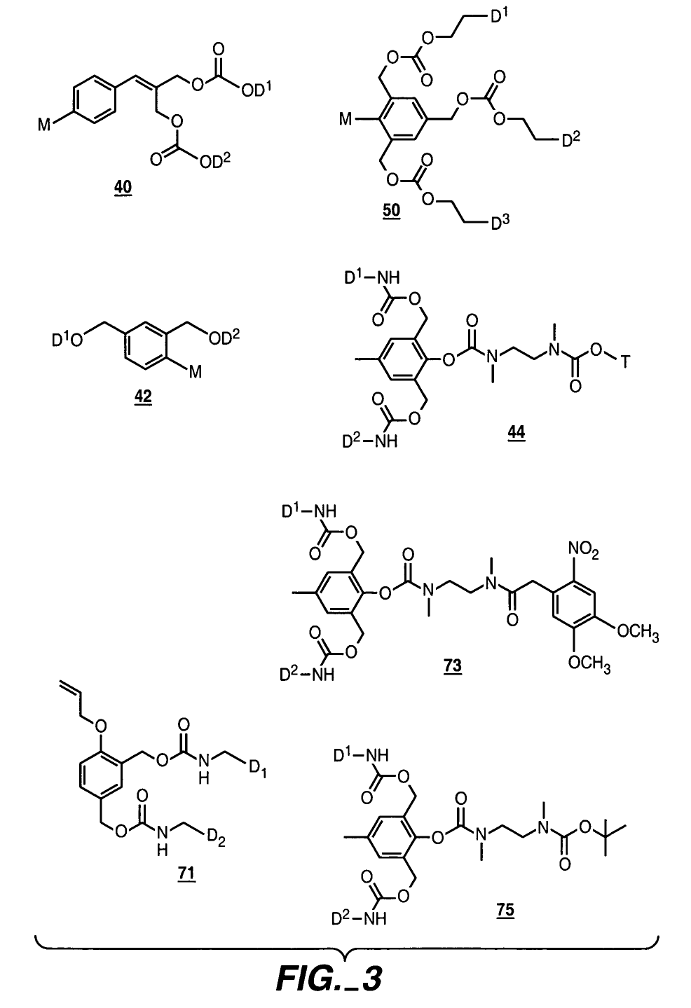

In some embodiments, the instant substrate compounds are not enzyme substrates, and can be used in assays of non-enzyme analytes. In some embodiments, the trigger moiety comprises an allyl trigger moiety and can be cleaved under conventional allyl deprotection conditions (Szalai et al., 2003). Exemplary substrate compound 71 comprises an allyl trigger moiety (FIG. 3).

In some embodiments, the trigger moiety comprises a photolabile trigger moiety (e.g., such as described by Amir et al. 2003), and can be cleaved upon irradiation with UV light. Exemplary substrate compound 73 comprises a photolabile trigger moiety.

In some embodiments, the trigger moiety comprises a tert-butoxycarbonyl moiety, as exemplified by substrate compound 75. In this embodiment, the triggering agent comprises trifluoroacetic acid.

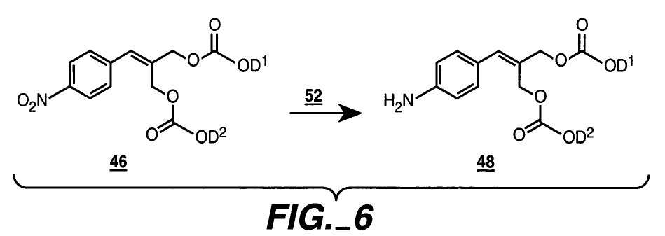

In some embodiments, cleavage of the trigger moiety by the specified triggering agent can initiate fragmentation of the self-immolative dendrimer linker indirectly via the formation of an intermediate compound. In these embodiments, the intermediate compound generates a π electron donor group that initiates a spontaneous reaction that results in the fragmentation of the multivalent linker. For example, the trigger moiety can comprise an aromatic nitro or azide group that can be reduced, thereby generating an electron donor group that is capable of initiating fragmentation of the self-immolative dendrimer linker and release of the fluorescent moiety(ies). Reaction 52 (FIG. 6) illustrates triggering of a nitroaromatic trigger moiety in substrate compound 46 by a chemical reduction to yield the aromatic amine 48. In this embodiment, the triggering agent comprises a reducing agent capable of carrying out the reduction reaction. An exemplary triggering agent comprises Zn and acetic acid (e.g., such as described by de Groot et al., 2003).

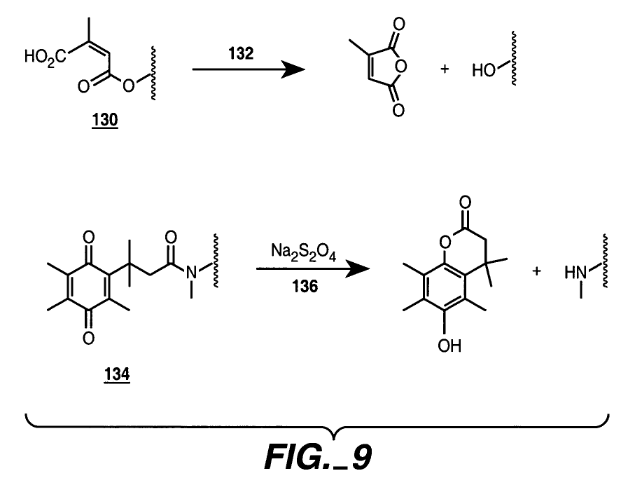

In some embodiments, the trigger moiety comprises a thermally labile trigger moiety (see, e.g., Carpino et al., 1993, Tetrahedron Lett. 34:7009-7012), and can be cleaved upon application of heat. Exemplary substrate compound 130 (FIG. 9) comprises a thermally labile trigger moiety which undergoes reaction 132 under conditions of elevated temperature.

In some embodiments, the trigger moiety comprises a chemically labile trigger. In some embodiments, such a trigger moiety comprises a redox-sensitive trigger moiety (see, e.g., Wang et al., 1995, J. Org. Chem. 60:539-543), and can be cleaved upon application of mild reduction conditions (e.g., Na₂S₂O₄ or electrochemically). Exemplary substrate compound 134 (FIG. 9) comprise a redox sensitive amine protective group which undergoes reaction 136 in the presence of Na₂S₂O₄.

As described hereinabove, the fragmentation of the self-immolative dendrimer linker comprises an elimination reaction. The particular elimination reaction will depend upon the chemical structure of the self-immolative dendrimer linker. In some embodiments, the core of the linker comprises a benzyl compound bearing, for example, a hydroxyl or amino substituent at the C1 carbon of the benzyl ring. The trigger moiety can be directly, or indirectly attached through spacers, to the hydroxyl or amino substituent. The self-immolative dendrimer linker can comprise any chemical structure to which the trigger moiety and tail units can be attached and which is capable of undergoing self-immolative fragmentation to release the fluorescent moiety(ies) from the reporter system upon triggering of the trigger moiety as described herein.

Additional embodiments of self-immolative dendrimer linkers suitable for use in the present methods, and synthetic methods therefor, are described in WO2004/019993. In some embodiments, self-immolative chemical linkers according to the present disclosure are five- or six-membered aromatic rings that have the following general formulas: 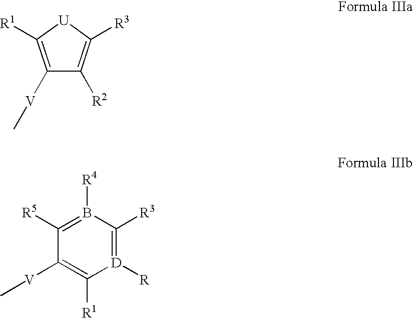
wherein:
V is O, S, PR⁶ or NR⁷;
U is O, S or NR⁸;
B and D are each independently a carbon atom or a nitrogen atom;
R¹, R², R³, R⁴ and R⁵ are each independently: 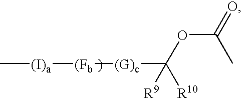
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, or alternatively, at least two of R¹, R², R³, R⁴ and R⁵ being connected to one another to form an aromatic or aliphatic cyclic structure;
   whereas:
a, b and c are each independently as integer of 0 to 5; and I, F and G are each independently —R¹¹C═CR¹²— or —C≡C—, where each of R¹¹ and R¹² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl alkylpiperazinyl, morpholino, tetrazole, carboxy, carboxylate, sulfoxy, sulfonate, sulfonyl, sulfixy, sulfinate, sulfinyl, phosphonooxy or phosphate, or, alternatively, R¹¹ and R¹² being connected to one another to form an aromatic or aliphatic cyclic structure; and
R⁶, R⁷ and R⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfonyl, phosphonooxy or phosphate,
   provided that at least two of R¹, R² and R³ in Formula IIIa and of R¹, R², R³, R⁴ and R⁵ in Formula IIIb are

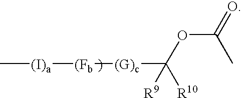

In some embodiments of self-immolative chemical linkers of the present disclosure, in the first generation of the dendrimer, V represents a group that links the chemical linker to the trigger, whereas in the advanced generations (n>1) V represents a group that links the linker to the chemical linkers of a previous generation. As is described hereinabove, V can be an etheric group (—O—), a thioetheric group (—S—), a substituted or non-substituted amino group (—NR⁶—) or a substituted or non-substituted phosphinic group (—PR⁷—).

In some embodiments of self-immolative chemical linkers of the present disclosure, the linker is linked to the tail units or to the linkers of the next generation via two or more groups:

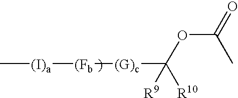

The —(I)a-(F)b-(G)_{c}—unit, if present, is a linear electronic cascade unit that is conjugated to the aromatic system of the basic unit and thereby directly participate in the self-immolative reactions sequence, whereas the carboxy unit —O—(C═O)— enables the release of the linkers/tail units attached thereto via a decarboxylation, which takes place at the end of the self-immolation sequence.

The presence of two or more such 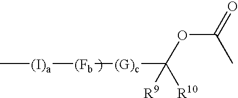
groups as substituents of the aromatic system enables the occurrence of more than one self-immolative reactions sequence at a time. The aromatic system, while being capable to undergo various rearrangements, further enables such occurrence. However, as such rearrangements are more facilitated in a six-membered aromatic ring, the chemical linker of the present disclosure can have the general formula IIIb.

Hence, at least two of the rings substituents R¹, R², R³, R⁴ and R⁵ in Formula IIIb are:

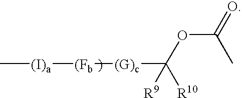

In some embodiments, at least two of R¹, R³ and R⁵ are:

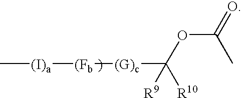

Other ring substituents, as well as the other substituents in Formulas IIIa and IIIb, R⁶, R⁷, R⁸, R⁹, R¹⁰, R¹¹ and R¹², can be hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate.

As used herein, the term “alkyl” refers to a saturated aliphatic hydrocarbon including straight chain and branched chain groups. In some embodiments, the alkyl group is a medium size alkyl having 1 to 10 carbon atoms, is a lower alkyl having 1 to 6 carbon atoms, or is an alkyl having 1 to 4 carbon atoms. Representative examples of an alkyl group are methyl, ethyl, propyl, isopropyl, butyl, tert-butyl, pentyl and hexyl.

As used herein, the term “cycloalkyl” refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane.

The term “aryl” refers to an all-carbon monocyclic or fused-ring polycyclic (i.e., rings which share adjacent pairs of carbon atoms) group having a completely conjugated pi-electron system. Examples, without limitation, of aryl groups are phenyl, naphthalenyl and anthracenyl.

The term “phenyl”, according to the present disclosure can be substituted by one to three substituents or non-substituted. When substituted, the substituent group may be, for example, halogen, alkyl, alkoxy, nitro, cyano, trihalomethyl, alkylamino or monocyclic heteroaryl.

The term “heteroaryl” includes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heterocycloalkyl” refers to a monocyclic or fused ring group having in the ring(s) one or more atoms such as nitrogen, oxygen and sulfur. The rings may also have one or more double bonds. However, the rings do not have a completely conjugated pi-electron system.

As used herein, the term “hydroxy” refers to an —OH group.

The term “thiohydroxy” refers to a —SH group.

The term “alkoxy” refers to both an —O-alkyl and an —O-cycloalkyl group, as defined herein below. Representative examples of alkoxy groups include methoxy, ethoxy, propoxy and tert-butoxy.

The term “thioalkoxy” refers to both a —S-alkyl and a —S-cycloalkyl group, as defined hereinabove.

The term “aryloxy” refers to both an —O-aryl and an —O-heteroaryl group, as defined herein.

A “thioaryloxy” group refers to both an —S-aryl and an —S-heteroaryl group, as defined herein.

As used herein, the term “halo” refers to a fluorine, chlorine, bromine or iodine atom.

The term “trihalomethyl” refers to a —CX₃ group, wherein X is halo as defined herein. A representative example of a trihalomethyl group is a —CF₃ group.

The term “amino” refers to an —NR′R″ group, where R′ and R″ are each independently hydrogen, alkyl or cycloalkyl, as is defined hereinabove.

The term “cyclic alkylamino” refers to an —NR′R″ group where R′ and R″ form a cycloalkyl.

The term “nitro” refers to a —NO₂ group.

The term “cyano” refers to a —C≡N group.

The term “C-amido” refers to a —C(═O)—NR′R″ group, where R′ and R″ are as described hereinabove.

The term “N-amido” refers to a —NR′—C(═O)—R″, where R′ and R″ are as described hereinabove.

The term “carboxy” refers to a —C(═O)—OH group.

The term “carboxylate” refers to a —C(═O)—OR′ group, where R′ is as defined hereinabove.

The term “sulfoxy” refers to a —S(═O)₂OH group.

The term “sulfonate” refers to a —S(═O)₂R′ group, where R′ is as defined herein above.

An “alkylsulfinyl” group refers to an —S(═O)—R′ group, where R′ is as defined herein.

The term “sulfonyl” refers to an —S(═O)₂—R′ group, where R′ is as defined herein.

The term “sulfixy” refers to an —S(═O)₂—H group.

The term “sulfinate” refers to an —S(═O)—OR′ group, where R′ is as defined hereinabove.

The term “sulfinyl” refers to an —S(═O)R′ group, where R′ is as defined hereinabove.

The term “phosphonooxy” refers to an —O—P(═O)(OH)₂ group.

The term “phosphate” refers to an —O—P(═O)(OR′)(OR″) group, where R′, and R″ are as defined hereinabove.

Alternatively, at least two of R¹, R², R³, R⁴ and R⁵ can be connected to one another, so as to form an aromatic or aliphatic cyclic structure. Thus, for example, the self-immolative linker comprises an aromatic system that include two or more fused rings (e.g., naphthalene or anthracene), or an aromatic ring that is fused to one or more alicyclic rings.

In some embodiments, a self-immolative linker according to the present disclosure has a general Formula IIIb, wherein V is O or S, each of B and D is a carbon atom, each of R² and R⁴ is hydrogen or alkyl, a, b and c are all 0 and R⁹ and R¹⁰ are hydrogen or alkyl.

In some embodiments of the present disclosure, the self-immolative dendrimers of the present disclosure further comprise a self-immolative spacer. As is well known in the art, the term “spacer” describes a residue, as is defined hereinabove, of a non-functional molecule, which is incorporated in a compound in order to facilitate its function and/or synthesis.

The spacer of the present disclosure may link the trigger moiety and/or one or more tail units to the chemical linker.

Incorporation of a self-immolative spacer between the chemical linker and the trigger moiety provides for and determines the distance there between. Such a distance is oftentimes required to facilitate the cleavage of the trigger moiety by rendering the trigger unhindered and non-rigid and thus exposed and susceptible to interact with the trigger.

Incorporation of a self-immolative spacer between a tail unit and the chemical linker is typically performed so as to facilitate the incorporation of a tail unit into the SID in terms of, for example, chemical compatibility and/or steric considerations.

Being selected as self-immolative, a spacer of the present disclosure participates in the self-immolative reactions sequence of the SIDs of the present disclosure.

In some embodiments, self-immolative spacers according to the present disclosure have a general formula selected from Formulas IVa, IVb, IVc and IVd below: 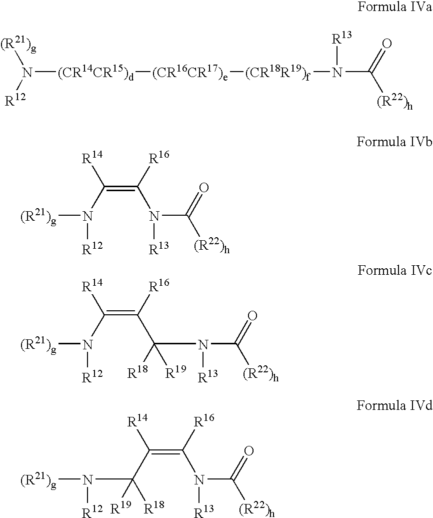
and a combination thereof,
wherein:
d, e, f, g and h are each independently an integer from 0 to 3, provided that d+e+f≧2;
R¹² and R¹³ are each independently hydrogen, alkyl or cycloalkyl;
R¹⁴, R¹⁵, R¹⁶, R¹⁷, R¹⁸ and R¹⁹ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate;
R²¹ and R²² each independently has a general formula selected from the group consisting of Formula Va and Formula Vb: 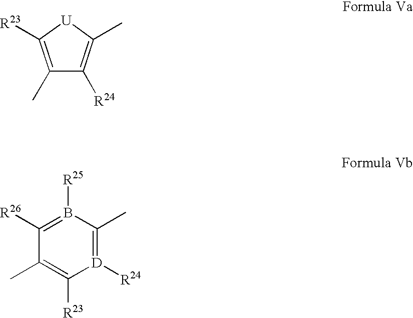
   wherein:
U is O, S or NR²⁹;
B and D are each independently a carbon atom or a nitrogen atom;
R²³, R²⁴, R²⁵ and R²⁶ are each independently: 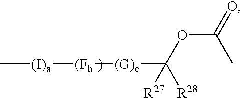
   hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove, or alternatively, at least two of R²³, R²⁴, R²⁵ and R²⁶ being connected to one another to form an aromatic or aliphatic cyclic structure;
   whereas:
a, b and c are each independently as integer of 0 to 5; and I, F and G are each independently —R³⁰C═CR³¹ or — C≡C—, where each of R³⁰ and R³¹ is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are described hereinabove, or, alternatively, R³⁰ and R³¹ being connected to one another to form an aromatic or aliphatic cyclic structure; and
R²⁹ is hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxy, carboxylate, sulfoxy, sulfonate, sulfonyl, sulfixy, sulfinate, sulfinyl, phosphonooxy or phosphate, as these terms are defined hereinabove,
provided that at least two of R²³ and R²⁴ in Formula Va and of R²³, R²⁴, R²⁵ and R²⁶ in Formula Vb are:

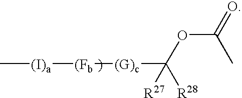

The spacers presented by Formulas IVa, IVb, IVc and IVd therefore belong to the known ω-amino aminocarbonyl cyclization spacers, which undergo self-elimination via a cyclization process, so as to form urea derivatives. By being terminated with an amine group, such spacers enable the formation of amide bonds.

As is described hereinabove, the self-immolative spacer of the present disclosure can also comprise any combination of the spacers presented in Formulas IVa, IVb, IVc and IVd, and, as is defined hereinabove, may further be interrupted with units that self-immolate via the electronic cascade self-elimination described hereinabove.

The chemical characteristics and the length of the self-immolative spacer can be tailored according to specific requirements, needs and/or preferences. For example, in cases where the tail units are large, bulky molecules and the reaction of the trigger moiety and the trigger requires unhindered trigger moiety (as in the case of enzymatic cleavage), a long self-immolative spacer may be incorporated in the SID, so as to avoid steric hindrance of the trigger moiety and hence, the selected spacer would comprise several, same or different, self-immolative spacer units. Also, in cases where the tail unit does not have a functional group that enables its attachment to the selected chemical linker, an appropriate spacer that can “divert” the functional group of the tail unit, can be incorporated.

Hence, the self-immolative dendrimers of the present disclosure are comprised of a cleavable trigger moiety, one or more self-immolative chemical linkers, a plurality of tail units and optionally one or more self-immolative spacers, all are attached one to the other in accordance with the unique dendrimeric structure.

The SIDs of the present disclosure can therefore be presented by Formula VI, as follows:
Q−Ai−Z⁰[(X₀)j(Y₀)k]−Z¹l[(X₁)l(Y₁)m]− . . . −Zⁿ[(Xₙ)p(Yₙ)r]−Zⁿ⁺¹[W] Formula VI
wherein:
n is an integer from 0 to 20;
each of i, j, k, l, m, p and r is independently an integer of 0 to 10;
Q is a cleavable trigger moiety, as is defined hereinabove;
A is a first self-immolative spacer, as is defined hereinabove;
Z is an integer of between 2 and 6, representing the ramification number of the dendrimer;
X is a self-immolative chemical linker, as is described hereinabove;
y is a second self-immolative spacer; and
W is a tail unit,
   whereas, when n equals 0, each of 1, m, p and r equals 0; and
   when n equals 1, each of p and r equals 0.

As has already been mentioned hereinabove, the ramification number of the SIDs of the present disclosure, represented by Z in Formula VI can be 2, 3, 4 or 5. The tail units W are defined and described herein.

In some embodiments, the number of generations of the SIDs, n, is 1-10 or 2-6.

The SIDs of the present disclosure can be easily designed, by selecting the appropriate linkages between the components, to be completely stable prior to contacting the trigger.

In some embodiments, the self-immolative dendrimer linker is based on a double release linker subunit. In some embodiments, the self-immolative dendrimer linker is based on a 2-(4-amino benzylidene)propane-1,3-diol linker subunit. Synthesis of this linker subunit, and various dendrimer compounds based on this subunit, is described by de Groot et al. (2003, Angew. Chem. Int. Ed. 42:4490-4494). FIG. 3 illustrates a first generation dendrimer compound 40 based on this linker subunit. Another embodiment of a dendrimer based on this subunit is shown at 46 and includes a nitroaromatic trigger moiety. In the structures shown in FIG. 3, “M” is a trigger moiety, and fluorescent moieties are represented as D¹, D² and D³. In some embodiments, M comprises a leaving group T (as described above) plus a π electron donor group, for example as shown in structure 58 (FIG. 4).

In another example, the linker is based on a 2,4-bis-(hydroxymethyl)phenol linker subunit. The synthesis of this subunit, and various dendrimer substrate compounds, is described by Szalai et al. (2003, J. Am. Chem. Soc. 125:15688-15689). An exemplary first generation dendrimer substrate compound based on this linker subunit is shown as structure 42. Another exemplary first generation dendrimer substrate compound based on this linker subunit is shown as structure 58 in FIG. 4. A second generation dendrimer substrate compound based on this linker subunit is shown as structure 70 in FIG. 5.

In another example, the linker is based on a 2,6-bis-(hydroxymethyl)-p-cresol linker subunit. Preparation of this subunit and dendrimer substrate compound is described by Amir et al. (2003, Angew. Chem. Int. Ed 42:4494-4499; and by Shamis et al. (2003)). An exemplary first generation dendrimer substrate compound based on this linker subunit is shown at 44. This compound is capable of undergoing a double release 1,4 elimination (see, e.g., Amir et al. (2003)).

In some embodiments, the linker is based on a triple release linker subunit. In one example, the linker is based on a (2-amino-3,5-di(hydroxymethyl)phenyl)methanol linker subunit (see, e.g., de Groot et al., 2003). Dendrimer substrate compound 50 is an exemplary first generation structure based on this linker subunit.

In another aspect of the instant disclosure, methods for detecting the activity of one or more triggering agents in a sample are provided. In some embodiments of the methods, a mixture is provided comprising a sample and a self-immolative dendrimer substrate, wherein the substrate comprises (a) a fluorescent reporter system, (b) a trigger moiety, (c) and a self-immolative dendrimer linker linking the reporter system to the trigger moiety. The fluorescent reporter system is at least partially quenched. The mixture is subjected to conditions effective to allow cleavage of the trigger moiety when the triggering agent is present in the sample, thereby increasing a fluorescent signal produced by the fluorescent reporter system. Detection of an increase in fluorescent signal in the mixture indicates the presence of the triggering agent in the sample.

In some embodiments, the triggering agent comprises an enzyme and the trigger moiety is a leaving group that is recognized and cleaved by the enzyme. The substrate can be designed to be reactive with a particular enzyme or a group of enzymes, or it can be designed to determine substrate specificity and other catalytic features, such as determining a value for kcat or Km.

The sample to be tested may be any suitable sample selected by the user. The sample may be naturally occurring or man-made. For example, the sample may be a blood sample, tissue sample, cell sample, buccal sample, skin sample, urine sample, water sample, or soil sample. The sample can be from a living organism, such as a eukaryote, prokaryote, mammal, human, yeast, or bacterium. The sample may be processed prior to contact with a substrate described herein by any method known in the art. For example, the sample may be subjected to a precipitation step, column chromatography step, heat step, etc. In some cases, the sample is a purified or synthetically prepared enzyme that is used to screen for or characterize an enzyme substrate, inhibitor, activator, or modulator.

If the sample contains two enzyme activities that may interfere (e.g., both a kinase and a phosphatase) so that the activity of one may interfere with the activity of the other, then an inactivating agent (e.g., an active site directed an irreversible inhibitor) can be added to the sample to inactivate whichever activity is not desired.

In some embodiments, the reaction mixture comprises a buffer, such as a buffer described in the “Biological Buffers” section of the 2000-2001 Sigma Catalog. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the enzyme to be detected. For example, the pH can be from 2 to 12, from 4 to 11, or from 6 to 10. The reaction mixture also contains any necessary cofactors and/or cosubstrates for the enzyme (e.g., ATP for a protein kinase, Ca²⁺ ion for a calcium dependent kinase, and cAMP for a protein kinase A).

In some embodiments, for example for substrate compounds comprising a non-enzyme cleavable trigger moiety, a reaction as described herein can be carried out in a partially or wholly non-aqueous medium, such as a conventional organic solvent. Non-limiting examples of such media include methanol, benzene, and DMSO.

As described above, the reaction product is more fluorescent than the substrate, so that a detectable increase in fluorescence can be observed. Generally, a greater change in fluorescence provides greater assay sensitivity, provided that an adequately low signal-to-noise ratio is achieved. Therefore, it may be desirable to test multiple substrate variants to find a substrate having the most suitable fluorescence properties.

The present disclosure contemplates not only detecting target enzymes, but also methods involving: (1) screening for and/or quantifying enzyme activity in a sample, (2) determining kcat and/or Km of an enzyme or enzyme mixture with respect to selected substrates, (3) detecting, screening for, and/or characterizing substrates of enzymes, (4) detecting, screening for, and/or characterizing inhibitors, activators, and/or modulators of enzyme activity, and (5) determining substrate specificities and/or substrate consensus sequences or substrate consensus structures for selected enzymes.

For example, in screening for enzyme activity, a sample that contains, or may contain, a particular enzyme activity is mixed with any of the substrates described herein, and the fluorescence is measured to determine whether an increase in fluorescence has occurred. Screening may be performed on numerous samples simultaneously in a multi-well or multi-reaction plate or device to increase the rate of throughput. Kcat and Km may be determined by standard methods, as described, for example, in Fersht, Enzyme Structure and Mechanism, 2nd Edition, W.H. Freeman and Co., New York, (1985)).

In some embodiments, the reaction mixture may contain two or more different enzymes, and may contain two or more different substrates. This may be useful, for example, to screen multiple enzymes simultaneously to determine if at least one of the enzymes is present or has a particular enzyme activity.

The substrate specificity of an enzyme can be determined by reacting an enzyme with different substrate compounds having different enzyme recognition moieties, and the activity of the enzyme toward the substrate compounds can be determined based on an increase in their fluorescence. For example, by reacting an enzyme with several different substrate compounds having several different trigger moieties (i.e., leaving groups), a consensus sequence for preferred substrates of an enzyme can be prepared.

Each different substrate compound may be tested separately in different reaction mixtures, or two or more substrate compounds may be present simultaneously in a reaction mixture. In embodiments in which the different substrate compounds are present simultaneously in the reaction mixture, the substrate compounds can contain the same fluorescent moiety, in which case the observed fluorescent signal is the sum of the signals from enzyme reaction with both substrates. Alternatively, the different substrate compounds can contain different, fluorescently distinguishable fluorescent moieties that allow separate monitoring and/or detection of the reaction of enzyme with each different substrate compound simultaneously in the same mixture.

Detecting, screening for, and/or characterizing inhibitors, activators, and/or modulators of enzyme activity can be performed by forming reaction mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant), IC₅₀, and the like, to characterize the concentration dependence of the effect that such substances have on enzyme activity. The effect of the modulator can be detected in various way. For example, when detecting a fluorescence signal in the presence of a putative modulator, a change in the fluorescence signal as compared to a control reaction or to a standard curve may indicate that the candidate modulator modulates the activity of the enzyme.

Detection of fluorescent signal can be performed in any appropriate way. Advantageously, any of the substrates described herein can be used in a continuous monitoring phase, in real time, to allow the user to rapidly determine whether enzyme activity is present in the sample, and optionally, the amount or specific activity of the enzyme. The fluorescent signal is measured from at least two different time points, usually until an initial velocity (rate) can be determined. The signal can be monitored continuously or at several selected time points. Alternatively, the fluorescent signal can be measured in an end-point embodiment in which a signal is measured after a certain amount of time, and the signal is compared against a control signal (before start of the reaction), threshold signal, or standard curve.

In the methods described herein, a fluorescence signal can be detected using conventional methods and instruments. In some embodiments, a multiwavelength fluorescence detector can be utilized. The detector can be used to excite the fluorescence labels at one wavelength and detect emissions at multiple wavelengths, or excite at multiple wavelengths and detect at one emission wavelength. Alternatively, the sample can be excited using “zero-order” excitation in which the full spectrum of light (e.g., from xenon lamp) illuminates the sample. Each label can absorb at its characteristic wavelength of light, or within an absorbance wavelength range, and then emit maximum fluorescence. The multiple emission signals can be detected independently. Preferably, a suitable detector can be programmed to detect more than one excitation emission wavelength substantially simultaneously, such as that commercially available under the trade designation HP1100 (G1321A) (Hewlett Packard, Wilmington, Del.). Thus, the fluorescent products can be detected at programmed emission wavelengths at various intervals during a reaction.

Also provided are kits for performing the various methods described herein. In some embodiments, the kit comprises at least one self-immolative dendrimer substrate compound, as described herein for detecting a triggering agent. The kit may include a buffer or other reagent for preparing a reaction mixture that facilitates the reaction.

In some embodiments, there are provided kits for detecting an enzyme activity. The kit can comprise one or more of the following: at least one self-immolatve dendrimer substrate compound as described herein, wherein the substrate is capable of producing a detectable signal when acted on by a triggering agent, such as an enzyme. In some embodiments, the enzyme is β-galactosidase and the substrate is a β-galactosidyl substituted self-immolative dendrimer compound or substituted derivative thereof. In some embodiments, the substrate comprises 9H-(1,3-dichloro-9,9-dimethylacridin-2-one-7-yl) β-D-galactopyranoside. In some embodiments, the enzyme is caspase 3 and the substrate compound comprises a substrate of caspase 3. In some embodiments, the enzyme is β-lactamase. The kit can include a modulator (e.g., an inhibitor or an activator) of the enzyme. In one example, an inhibitor of β-galactosidase, β-lactamase or of capase 3 is included. The kit may include a buffer or other reagent. The choice of a particular buffer may depend on various factors, such as the pH optimum for the enzyme to be detected, the solubility and fluorescence properties of the fluorescent moiety in the substrate, and the pH of the sample from which the target enzyme is obtained. The buffer is usually added to the reaction mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the enzyme assay, as discussed above. The pH of the reaction mixture may also be titrated with acid or base to reach a final, desired pH. The kit may additionally include other components that are beneficial to enzyme activity, such as salts (e.g., KCl, NaCl, or NaOAc), metal salts (e.g., Ca²⁺ salts such as CaCl₂, MgCl₂, MnCl₂, ZnCl₂, or Zn(OAc), detergents (e.g., TWEEN 20), and/or other components that may be useful for a particular enzyme. These other components can be provided separately from each other or mixed together in dry or liquid form. The enzyme substrate compound can also be provided in dry or liquid form, together with or separate from the buffer. To facilitate dissolution in the reaction mixture, the enzyme substrate compound can be provided in an aqueous solution, partially aqueous solution, or non-aqueous stock solution that is miscible with the other components of the reaction mixture. For example, in addition to water, a substrate solution may also contain a cosolvent such as dimethyl formamide, dimethylsulfonate, methanol or ethanol, typically in a range of 1%-10% (v:v).

A kit can also include instructional material for carrying out embodiments of the methods as described herein.

Unless defined otherwise, all technical and scientific terms used herein have the same meaning as commonly understood by one of ordinary skill in the art to which compositions and methods belong. Unless mentioned otherwise the techniques employed or contemplated herein are standard methodologies well known to one of ordinary skill in the art. The materials, methods and examples are illustrative only and not limiting.

All numerical ranges in this specification are intended to be inclusive of their upper and lower limits.

All patent applications, patents, and literature references cited in this specification are hereby incorporated by reference in their entirety. In case of conflict or inconsistency, the present description, including definitions, will control.

### 7. EXAMPLES

7.1 Preparation of a Self-Immolative Dendrimer Substrate

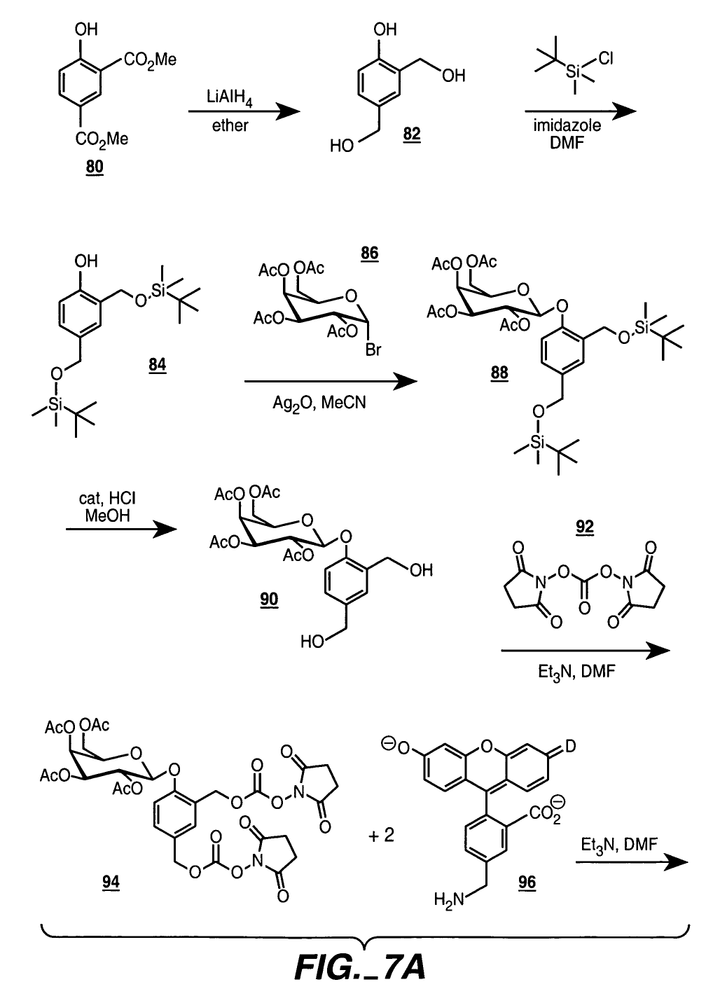

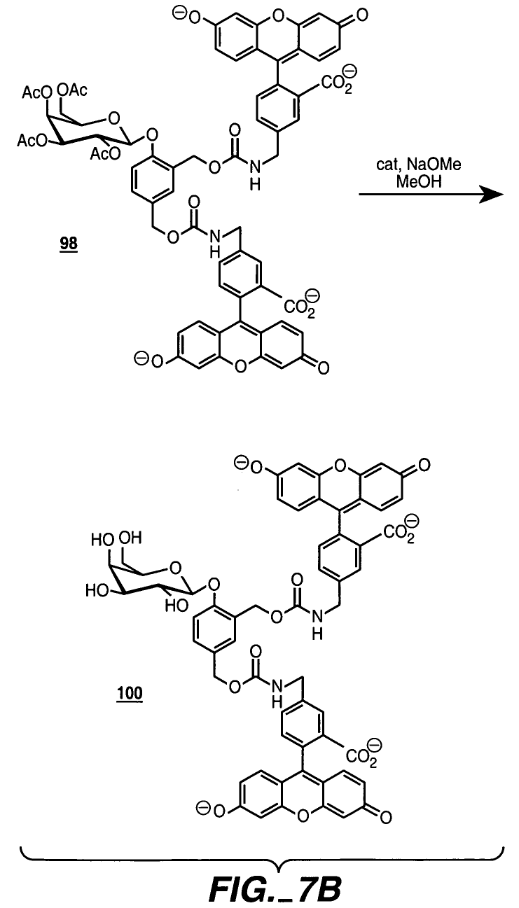

Referring to FIGS. 7A-7B Dimethyl 4-hydroxyisophthalate (80, Aldrich 54,109-5) is reduced with lithium aluminum hydride in refluxing ether to give triol 82. The benzylic alcohols are selectively protected with tert-butyl dimethylsilyl chloride and imidazole in dimethylformamide (DMF) to give compound 84. Glycosylation of 84 with 86 and silver(I) oxide in acetonitrile affords glycoside 88. Desilation is performed with catalytic acid in methanol to give diol 90. Treatment of compound 90 with disuccinimidylcarbonate (92) and triethylamine (TEA) in DMF affords 94. Coupling of 2 equivalents of aminomethylfluorescein (96) with 94 in DMF/TEA gives 98. Deprotection of the acetates is performed with catalytic sodium methoxide in methanol to afford self-immolative dendrimer substrate 100.

While the foregoing has presented specific embodiments, it is to be understood that these embodiments have been presented by way of example only. It is expected that others will perceive and practice variations which, though differing from the foregoing, do not depart form the spirit and scope of the teachings as described and claimed herein.

We claim:

1. A compound comprising:
a trigger moiety;
a reporter system comprising at least one fluorescent moiety; and
a multivalent self-immolative dendrimer linker linking said trigger moiety to said reporter system, wherein the fluorescence of the reporter system is at least partially quenched, and wherein said linker is capable of fragmenting to release said fluorescent moiety when said trigger moiety is acted upon by a triggering agent, leading to a detectable increase in a fluorescence signal.

2. The compound of claim 1 wherein said trigger moiety comprises a cleavage site for a cleaving enzyme.

3. The compound of claim 2 in which the cleaving enzyme is selected from a lipase, an esterase, a phosphatase, a protease, a glycosidase, a carboxypeptidase and a catalytic antibody.

4. The compound of claim 1 wherein said trigger moiety comprises an azide moiety.

5. The compound of claim 1 wherein said trigger moiety comprises a nitro moiety.

6. The compound of claim 1 wherein said trigger moiety comprises an allyl group.

7. The compound of claim 1 wherein said trigger moiety is selected from a photolabile moiety, a chemically labile moiety, or a thermally labile moiety.

8. The compound of claim 1 in which the reporter system comprises a fluorescent moiety and a quenching moiety.

9. The compound of claim 1 wherein the reporter system comprises a plurality of identical fluorescent moieties, and wherein said fluorescent moieties comprise self-quenching dyes.

10. The compound of claim 1 wherein said fluorescent moiety comprises a dye selected from a xanthene dye, a rhodamine dye, a fluorescein dye, a cyanine dye, a phthalocyanine dye, a squaraine dye and a bodipy dye.

11. The compound of claim 1 wherein said reporter system comprises a fluorescence donor moiety and a fluorescence acceptor moiety.

12. The compound of claim 1 comprising N_{b}^{G} tail units, where N_{b} is the branching multiplicity, and G is the generation of the compound.

13. The compound of claim 1 which comprises a first- or higher generation dendrimer compound based on the following structure: 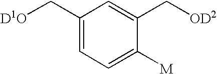
wherein at least one of D¹ and D² comprises a fluorescent moiety,
and wherein M comprises a trigger moiety.

14. The compound of claim 1 which comprises a first- or higher generation dendrimer compound based on the following structure: 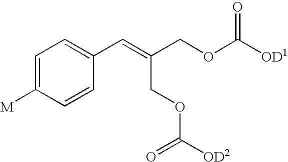
wherein at least one of D¹ and D² comprises a fluorescent moiety,
and wherein M comprises a trigger moiety.

15. The compound of claim 1 which comprises a first- or higher generation dendrimer compound based on the following structure: 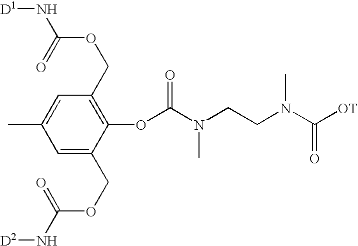
wherein at least one of D¹ and D² comprises a fluorescent moiety,
and wherein T comprises a leaving group recognized by an enzyme.

16. The compound of claim 1 in which the multivalent linker is capable of fragmenting via an elimination reaction.

17. The compound of claim 16 in which the elimination comprises 1,4-elimination.

18. The compound of claim 16 in which the elimination comprises 1,6-elimination.

19. The compound of claim 16 in which the fragmentation comprises a 1,8-elimination.

20. The compound of claim 1 wherein said linker is a double-release linker or a triple-release linker.

21. A kit comprising a compound according to claim 1 and at least one reagent.

22. A kit according to claim 21 in which said reagent comprises an enzyme capable of triggering said trigger moiety of said compound.

23. A kit according to claim 21 in which said reagent comprises a modulator of an enzyme.

24. A kit according to claim 21 wherein said trigger moiety comprises an enzyme substrate leaving group.

25. A compound comprising:
a trigger moiety;
a reporter system comprising at least one fluorescent moiety; and
a multivalent self-immolative dendrimer linker linking said trigger moiety to said reporter system, wherein the fluorescence of the reporter system is at least partially quenched, and wherein said linker is capable of fragmenting to release said fluorescent moiety when said trigger moiety is acted upon by a triggering agent, leading to a detectable increase in a fluorescence signal,
wherein the reporter system comprises a plurality of identical fluorescent moieties, and wherein said fluorescent moieties comprise self-quenching dyes.

26. A method of detecting the presence of a triggering agent in a sample, comprising the steps of:
contacting the sample with a substrate according to claim 1 under conditions effective to allow a triggering agent to trigger said trigger moiety, and
detecting a fluorescence signal, where an increase in the fluorescence signal indicates the presence and/or quantity of the triggering agent in the sample.

27. The method of claim 26 wherein said triggering agent comprises an enzyme and said trigger moiety comprises a cleavable enzyme substrate leaving group.

28. The method of claim 26 wherein said triggering agent comprises an enzyme and said trigger moiety comprises a cleavage site for an enzyme.

29. A method of identifying a modulator of an enzyme activity, comprising the steps of:
contacting the enzyme with a substrate according to claim 1 in the presence of a candidate modulator and under conditions effective to allow the enzyme to cleave said substrate; and
detecting a fluorescence signal, where a change in the fluorescence signal as compared to a control reaction or to a standard curve indicates that the candidate modulator modulates the activity of the enzyme.
